(19) United States
(12) Patent Application Publication
(10) Pub. No.: US 2024/0327678 A1
(43) Pub. Date: Oct. 3, 2024

(54) HYDROPHILICALLY MODIFIED SILICONE PRESSURE SENSITIVE ADHESIVE

(71) Applicant: MOMENTIVE PERFORMANCE MATERIALS INC., Niskayuna, NY (US)

(72) Inventors: Meenal Mehra, New Delhi (IN); Sandeep Naik, Maharashtra (IN); Tiberiu Mircea Siclovan, Rexford (NY); Arindam Sannigrahi, Bangalore (IN); Debarshi Dasgupta, Bangalore (IN); Praveen Mishra, Bangalore (IN); Sukanta Dolai, Bangalore (IN)

(21) Appl. No.: 18/128,688

(22) Filed: Mar. 30, 2023

(51) Int. Cl. C09J 7/38; C08G 77/46

(52) U.S. Cl. CPC C09J 7/38; C08G 77/46; C08G 77/70; C09J 2301/302; C09J 2301/414; C09J 2483/00

(57) ABSTRACT

A novel hydrophylically modified silicone and a composition for forming a hydrophylically modified silicone pressure sensitive adhesive is shown and described herein. The comprises comprises (i) a MQ silicone resin, (ii) a polyorganosiloxane, and (iii) a siloxane comprising a range of hydrophilic functional groups. The mixture of (i), (ii), and (iii) is self-catalyzing and does not require an external condensation catalyst. The composition is suitable for forming a cured silicone material such as, for example, a pressure sensitive adhesive compatible with polar additives or actives used in various applications.

### FIELD OF INVENTION

The present invention relates to a siloxane comprising a hydrophilic group, a silicone composition comprising the siloxane and suitable for forming a silicone pressure sensitive adhesive. In particular, the present invention relates to a composition that may be considered self-catalyzing, and a pressure sensitive adhesive materials formed from such compositions.

### BACKGROUND

Silicone pressure sensitive adhesives may be utilized in a variety of applications. One area where silicone pressure sensitive adhesives are finding more utility is personal care and healthcare applications. These can include many different types of products including, but not limited to, tapes, dressings, wearable devices, and the like. In such applications, the pressure sensitive adhesive is often in direct contact with the skin of a person wearing the product or device. Silicone pressure sensitive adhesives and gels can provide improved feel during wear as well as atraumatic removal compared to non-silicone based pressure sensitive adhesives.

While silicone pressure sensitive adhesives can provide benefits with respect to comfort, silicone pressure sensitive adhesives are hydrophobic materials. The high degree of hydrophobicity can limit how the adhesive may be used in personal care and healthcare applications. For example, the high degree of hydrophobicity may limit the compatibility of the adhesive with ingredients used in personal care and healthcare applications. Some prior attempts to overcome these issues have been to use non-silicone, polar materials as adhesives. Another option is to utilize additives to impart polar or hydrophilic characteristics to silicone adhesives. Non-reactive additives, however, are extractable or may migrate, which can change the adhesives composition and properties over time. Further, the use of additives to impart hydrophilicity, and in particular the amount of additives that can be used, is limited to an amount that will be miscible in the composition.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure.

Provided is a silicone composition and materials produced from such compositions. The silicone composition may be suitable for producing a silicone pressure sensitive adhesive. In accordance with aspects of the invention, the silicone composition comprises a silicone resin, a polyorganosiloxane, and a siloxane comprising a hydrophilic group selected from an ionic group and/or a zwitterionic group. The hydrophilic functional silicone material can be selected from a silicone resin (e.g., a MQ type resin) and/or a polyorganosiloxane functionalized with the hydrophilic group.

The silicone compositions are suitable for producing a silicone material such as, for example, a silicone pressure sensitive adhesive that has improved hydrophilicity compared to other silicone pressures sensitive adhesives while retaining desirable adhesive properties. In particular, the present compositions provide a silicone adhesive composition that includes a silicone with hydrophilic groups covalently bonded thereto. These groups may also be considered polar functionalities. The siloxane comprising a hydrophilic group have been found to improve the hydrophilicity of otherwise hydrophobic silicone adhesives.

In one aspect, provided is a siloxane comprising a hydrophilic group, the siloxane of the formula:

M¹ₐM²_{b}M³_{c}D¹_{d}D²ₑD³_{f}T¹_{g}T²ₕT³ᵢQ¹ⱼQ²ₖQ³ₗQ⁴ₒQ⁵ₚ
where
M¹=R⁵R⁶R⁷SiO_{1/2}
M²=R⁸R⁹R¹⁰SiO_{1/2}
M³=R¹¹R¹²R¹³SiO_{1/2}
D¹=R¹⁴R¹⁵SiO_{2/2}
D²=R¹⁶R¹⁷SiO_{2/2}
D³=R¹⁸R¹⁹SiO_{2/2}
T¹=R²⁰SiO_{3/2}
T²=R²¹SiO_{3/2}
T³=R²²SiO_{3/2}
Q¹=Si(OR²³)₁O_{3/2}
Q²=Si(OR²⁴)₂O_{2/2}
Q³=Si(OR²⁵)₃O_{1/2}
Q⁴=Si(OR²⁶)₄
Q⁵=SiO_{4/2}
where:
a, b, c, d, e, f, g, h, i, j, k, l, o, and p are zero or any positive integer subject to the following limitations:

$$1 \leq [ a + b + c + d + e + f + g + h + i + j + k + l + o + p ] \leq 6000 ;$$

$$[ b + e + h + k ] \geq 1 ;$$

R⁵, R⁶, R⁷, R⁹, R¹⁰, R¹², R¹³, R¹⁴, R¹⁵, R¹⁷, R¹⁹, and R²⁰ are each independently selected from hydrogen, a C1-C60 alkyl, a C6-C60 aromatic containing group, a C1-C10 alkoxy, and a hydroxyl; R⁸, R¹⁶, R²¹, and R²⁴ are each independently selected from a monovalent hydrophilic group of the formula, where G≥0.

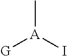

where A is a bridging unit between the siloxane moiety and the hydrophilic group I, having at least one spacing atom selected from a C1-C60 acyclic hydrocarbon radical, a C4-C60 alicylic radical, an C6-C60 aromatic hydrocarbon radical, a polyetheramine, a polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof,
I is an ionic, ionizable, zwitterionic group, or a polar hydrophilic group or moieties including but not limited to a polyetheramine, a polyether, a polyamide, a polyester, a polyurethane, a polysulfone, or a polycarbonate;
G is a polymerizable group having the general Formula:

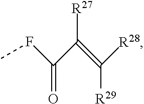

or
a non-polymerizable group of formula —F—K, wherein F is a linker group chosen from a C1-C60 acyclic hydrocarbon radical, a C4-C60 alicylic radical, an C6-C60 aromatic hydrocarbon radical, a polyetheramine, a polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof;
R²⁷, R²⁸, and R²⁹ are independently chosen from hydrogen or monovalent hydrocarbon radical with 1-5 carbon atoms, and
K is independently chosen from hydrogen, a C1-C60 acyclic hydrocarbon radical, a C4-C60 alicylic radical, an C6-C60 aromatic hydrocarbon radical, an epoxy, or an ether group;
R¹¹, R¹⁸, R²² are each independently selected from the group -A-G where A and G are as described above; and
R²³, R²⁵, and R²⁶ can be independently selected from any of R⁵ to R²² or a combination of thereof.

In one embodiment, the hydrophilic group is a polyetheramine group selected from a compound of the formula

R³⁶—(O—R³⁷—)_{q}—NH₂
where R³⁶ is selected from a C1-C60 hydrocarbon, R³⁷ is selected from a C1-C60 hydrocarbon, and q is ≥1.

In one embodiment in accordance with the siloxane of any of the previous embodiments, the polyetheramine is selected from a compound of the formula:

R³⁶—(O—CH₂CH₂)ₓ—(O—CCH₂CH₂CH₂)_{y}—(O CH₂CH₂CH(CH₃))_{z}—NH₂
and x, y, and z are independently 0 to about 300, where x+y+z is ≥2.

In one embodiment in accordance with the siloxane of any of the previous embodiments, the polyetheramine group is selected by the following general formula

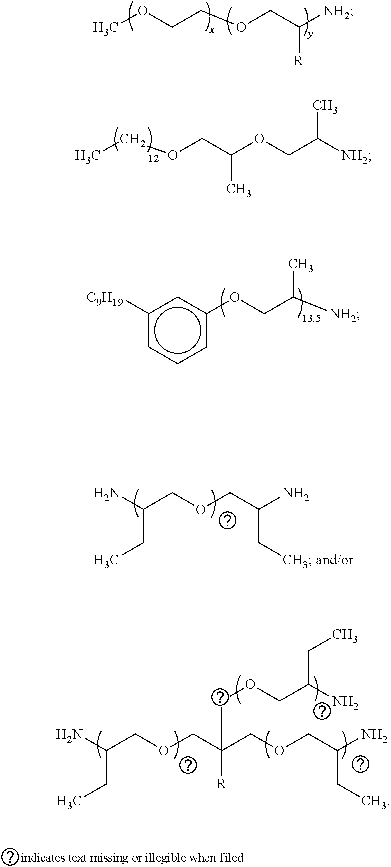

In one embodiment, the siloxane comprising a hydrophilic group is of the formula M¹ₐM²_{b}M³_{c}Q⁵ₚ, where b is ≥1.

In one embodiment in accordance with the siloxane of any of the previous embodiments, I is selected from a carboxylate —COO⁻, a dicarboxylate (—R(COO—)₂) a sulfone SO₂—, a sulfonate SO₃⁻, a sulfate —OSO₃⁻, a phosphonate —PO₃²⁻, a phosphate OPO₃²⁻ group, —N⁺R³⁰R³¹H, —N⁺H₂R³², —N⁺H₃, or an ammonium salt, each containing a hydrogen or a cation independently selected from an alkali metal, an alkali earth metal, a transition metal, a quaternary ammonium group, and a phosphonium group, where R³⁰, R³¹, and R³² are independently selected from a C1-C30 hydrocarbon.

In one embodiment in accordance with the siloxane of any of the previous embodiments, I is selected from a group having the formula —R³³—N+(R³⁴)₂—R³⁵—I^{z} where R³³ is a divalent hydrocarbon group having from 1 to 20 carbon atoms, R³⁴ is a monovalent hydrocarbon group having from 1 to 20 carbon atoms, R³⁵ is a divalent hydrocarbon group having from 2 to 20 carbon atoms; and I^{z} is an ionic group selected from a carboxylate —COO⁻, a sulfone —SO₂—, a sulfonate —SO₃⁻, a sulfate —OSO₃⁻, a phosphonate —PO₃²⁻, and a phosphate —OPO₃²⁻ group.

In another aspect, provided is a composition for forming a silicone pressure sensitive adhesive comprising (i) a MQ silicone resin, (ii) a polyorganosiloxane, and (iii) a siloxane comprising a hydrophilic group.

In one embodiment, the siloxane comprises a hydrophilic functional group, wherein the hydrophilic functional group is selected from an ionic group, an ionizable group, a zwitterionic group, a polyetheramine group, or a combination of two or more thereof.

In one embodiment in accordance with the composition of any of the previous embodiments, the siloxane comprising a hydrophilic group (iii) is selected from a compound of the formula:

M¹ₐM²_{b}M³_{c}D¹_{d}D²ₑD³_{f}T¹_{g}T²ₕT³ₗQ¹_{J}Q²ₖQ³ₗQ⁴ₒQ⁵ₚ
where
   M¹=R⁵R⁶R⁷SiO_{1/2}
   M²=R⁸R⁹R¹⁰SiO_{1/2}
   M³=R¹¹R¹²R¹³SiO_{1/2}
   D¹=R¹⁴R¹⁵SiO_{2/2}
   D²=R¹⁶R¹⁷SiO_{2/2}
   D³=R¹⁸R¹⁹SiO_{2/2}
   T¹=R²⁰SiO_{3/2}
   T²=R²¹SiO_{3/2}
   T³=R²²SiO_{3/2}
   Q¹=Si(OR²³)₁O_{3/2}
   Q²=Si(OR²⁴)₂O_{2/2}
   Q³=Si(OR²⁵)₃O_{1/2}
   Q⁴=Si(OR²⁶)₄
   Q⁵=SiO_{4/2}
   where:
   a, b, c, d, e, f, g, h, i, j, k, l, o, and p are zero or any positive integer subject to the following limitations:
   1≤[a+b+c+d+e+f+g+h+i+j+k+l+o+p]≤6000; [b+e+h+k]≥1;
   R⁵, R⁶, R⁷, R⁹, R¹⁰, R¹², R¹³, R¹⁴, R¹⁵, R¹⁷, R¹⁹, and R²⁰ are each independently selected from hydrogen, a C1-C60 alkyl, a C6-C60 aromatic containing group, a C1-C10 alkoxy, and a hydroxyl;
   R⁸, R¹⁶, R²¹, and R²⁴ are each independently selected from a monovalent hydrophilic group of the formula, where G≥0.

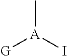

where A is a bridging unit between the siloxane moiety and the hydrophilic group I, having at least one spacing atom selected from a C1-C60 acyclic hydrocarbon radical, a C4-C60 alicylic radical, an C6-C60 aromatic hydrocarbon radical, a polyetheramine, a polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof, I is an ionic, ionizable, zwitterionic group, or a polar hydrophilic group;
G is a polymerizable group having the general Formula:

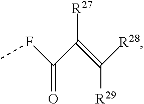

a non-polymerizable group of formula —F—K, wherein F is a linker group chosen from a C1-C60 acyclic hydrocarbon radical, a C4-C60 alicylic radical, an C6-C60 aromatic hydrocarbon radical, a a polyetheramine, polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof,
R²⁷, R²¹, and R²⁹ are independently chosen from hydrogen or monovalent hydrocarbon radical with 1-5 carbon atoms, and
K is independently chosen from hydrogen, a C1-C60 acyclic hydrocarbon radical, a C4-C60 alicylic radical, an C6-C60 aromatic hydrocarbon radical, an epoxy, or an ether group;
R¹¹, R¹⁸, R²² are each independently selected from the group -A-G where A and G are as described above; and
R²³, R²⁵, and R²⁶ can be independently selected from R⁵ to R²² or a combination of thereof.

In one embodiment, the siloxane comprising a hydrophilic group is of the formula M¹ₐM²_{b}M³_{c}Q⁵ₚ, where b is ≥1.

In one embodiment in accordance with the composition of any of the previous embodiments, I is selected from a carboxylate —COO⁻, a dicarboxylate (—R(COO—)₂) a sulfone —SO₂—, a sulfonate —SO₃⁻, a sulfate —OSO₃⁻, a phosphonate —PO₃²⁻, a phosphate —OPO₃²⁻ group, —N⁺R³⁰R³¹H, —N⁺H₂R³², —N⁺H₃, or an ammonium salt, each containing a hydrogen or a cation independently selected from an alkali metal, an alkali earth metal, a transition metal, a quaternary ammonium group, and a phosphonium group, where R³⁰, R³¹, and R³² are independently selected from a C1-C30 hydrocarbon.

In one embodiment in accordance with the composition of any of the previous embodiments, I is selected from a group having the formula —R³³—N⁺(R³⁴)₂—R³⁵—I^{z} where R³³ is a divalent hydrocarbon group having from 1 to 20 carbon atoms, R³⁴ is a monovalent hydrocarbon group having from 1 to 20 carbon atoms, R³⁵ is a divalent hydrocarbon group having from 2 to 20 carbon atoms; and I^{z} is an ionic group selected from a carboxylate —COO⁻, a sulfone —SO₂—, a sulfonate —SO₃⁻, a sulfate —OSO₃⁻, a phosphonate —PO₃²⁻, and a phosphate OPO₃²⁻ group.

In one embodiment in accordance with the composition of any of the previous embodiments, I is a polar group selected from a polyetheramine, a polyether, a polyamide, a polyester, a polyurethane, a polysulfone, and/or a polycarbonate.

In one embodiment in accordance with the composition of any of the previous embodiments, the hydrophilic group is a polyetheramine group selected from a compound of the formula

R³⁶—(O—R³⁷—)_{q}—NH₂
where R³⁶ is selected from a C1-C60 hydrocarbon, R³⁷ is selected from a C1-C60 hydrocarbon, and q is ≥1.

In one embodiment, the polyetheramine is selected from a compound of the formula:

R³⁶—(O—CH₂CH₂)ₓ—(O—CCH₂CH₂CH₂)_{y}—(O CH₂CH₂CH(CH₃))_{z}—NH₂
and x, y, and z are independently 0 to about 300, where x+y+z is ≥2.

In one embodiment in accordance with the composition of any of the previous embodiments, the polyetheramine group is selected by the following general formula

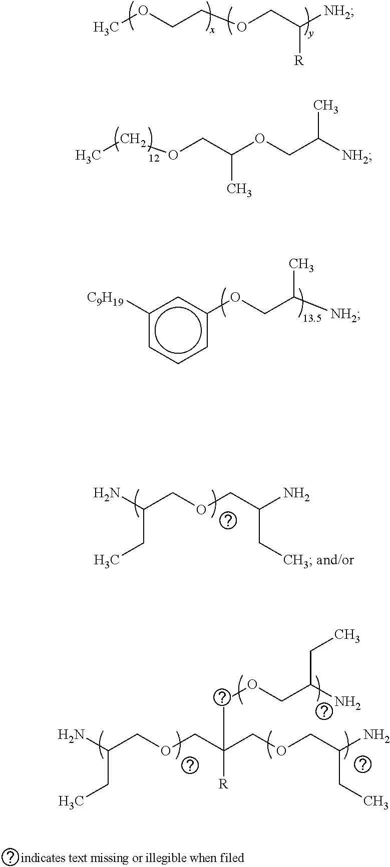

In one embodiment in accordance with the composition of any of the previous embodiments, the siloxane comprising a hydrophilic group (iii) is present in an amount of from about 0.1 wt. % to about 20 wt. % based on the total weight of the composition.

In one embodiment in accordance with the composition of any of the previous embodiments, the siloxane comprising a hydrophilic group (iii) is present in an amount of from about 1.5 wt. % to about 8 wt. % based on the total weight of the composition.

In still another aspect, provided is a pressure sensitive adhesive formed from the composition of any of the previous aspects and embodiments.

In one embodiment, the adhesive has a less than 2500 ppm of one or more of octamethylcyclotetrasiloxane, decamethylcyclopentasiloxane, and/or dodecamethylcyclohexasiloxane.

In one embodiment, the adhesive has a less than 1000 ppm of one or more of octamethylcyclotetrasiloxane, decamethylcyclopentasiloxane, and/or dodecamethylcyclohexasiloxane.

In one embodiment, the adhesive has a less than 500 ppm of one or more of octamethylcyclotetrasiloxane, decamethylcyclopentasiloxane, and/or dodecamethylcyclohexasiloxane.

In one embodiment, the adhesive has a less than 100 ppm of one or more of octamethylcyclotetrasiloxane, decamethylcyclopentasiloxane, and/or dodecamethylcyclohexasiloxane.

In yet another aspect, provided is an article comprising the pressure sensitive adhesive of any of the previous aspects or embodiments.

In one embodiment, the article is selected from a wound dressing, a dressing for scar reduction, a drug delivery device, a medical tubing, a clinical surface, a pacemaker lead, a wound healing patch, a wound management device, a catheter, a shunt, a valve, a stent, a transdermal patch, a scaffold for tissue engineering, an anti-microbial device, a ophthalmic device, a bioinsert, a plug, a surgical device, an assisted breathing apparatus, a prosthetic device, a reconstructive device, or a body implant.

In one embodiment, the article is selected from a cream, a lotions, a bioactive compound, deodorant, an insecticide, a decorative cosmetic, lipstick, eye shadow, stage make-up, a hand cleaner, a face mask, a pore cleanser, a knee- or elbow-protector, a bra, a surgical gown, a nasal plasters; a prosthesis, a cold wrap, a thermal wrap to a hearing aid; a protective face masks, an anti-snoring patch, jewelry, earrings, guises, tattoos, eye wear, a tape, or a bandage.

The following description and the drawings disclose various illustrative aspects. Some improvements and novel aspects may be expressly identified, while others may be apparent from the description.

### DETAILED DESCRIPTION

Reference will now be made to exemplary embodiments, examples of which are illustrated in the accompanying drawings. It is to be understood that other embodiments may be utilized and structural and functional changes may be made. Moreover, features of the various embodiments may be combined or altered. As such, the following description is presented by way of illustration only and should not limit in any way the various alternatives and modifications that may be made to the illustrated embodiments. In this disclosure, numerous specific details provide a thorough understanding of the subject disclosure. It should be understood that aspects of this disclosure may be practiced with other embodiments not necessarily including all aspects described herein, etc.

As used herein, the words “example” and “exemplary” means an instance, or illustration. The words “example” or “exemplary” do not indicate a key or preferred aspect or embodiment. The word “or” is intended to be inclusive rather than exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

It will be appreciated that ranges for a particular component can be combined to form new and non-specified ranges.

As used herein, the term, “hydrocarbon radical” generically refers to acyclic, cyclic (or alicyclic), and aromatic hydrocarbons which can be saturated or unsaturated and which may be saturated or unsaturated and which may be optionally substituted or interrupted with one or more atoms or functional groups, such as, for example, carboxyl, cyano, hydroxy, halo and oxy. It will be appreciated that the term can encompass monovalent, divalent, and trivalent radicals, and that the appropriate type of radical is indicated and intended in the context of a given formula or structure.

As used herein, the term “acyclic hydrocarbon radical” means a straight chain or branched hydrocarbon radical, preferably containing from 1 to 60 carbon atoms per radical, which may be saturated or unsaturated and which may be optionally substituted or interrupted with one or more atoms or functional groups, such as, for example, carboxyl, cyano, hydroxy, halo and oxy. Suitable monovalent acyclic hydrocarbon radicals include, for example, alkyl, alkenyl, alkynyl, hydroxyalkyl, cyanoalkyl, carboxyalkyl, alkyloxy, oxaalkyl, alkylcarbonyloxaalkylene, carboxamide and haloalkyl, such as, for example, methyl, ethyl, sec-butyl, tert-butyl, octyl, decyl, dodecyl, cetyl, stearyl, ethenyl, propenyl, butynyl, hydroxypropyl, cyanoethyl, butoxy, 2,5,8-trioxadecanyl, carboxymethyl, chloromethyl and 3,3,3-fluoropropyl.

As used herein, the term “alicyclic hydrocarbon radical” means a radical containing one or more saturated hydrocarbon rings, preferably containing from 4 to 12 carbon atoms per ring, per radical which may optionally be substituted on one or more of the rings with one or more alkyl radicals, each preferably containing from 2 to 6 carbon atoms per alkyl radical, halo radicals or other functional groups and which, in the case of a monovalent alicyclic hydrocarbon radical containing two or more rings, may be fused rings. Suitable monovalent alicyclic hydrocarbon radicals include, for example, cyclohexyl and cyclooctyl.

As used herein, the term “aromatic hydrocarbon radical” means a hydrocarbon radical containing one or more aromatic rings per radical, which may, optionally, be substituted on the aromatic rings with one or more alkyl radicals, each preferably containing from 2 to 6 carbon atoms per alkyl radical, halo radicals or other functional groups and which, in the case of a monovalent aromatic hydrocarbon radical containing two or more rings, may be fused rings. Suitable monovalent aromatic hydrocarbon radicals include, for example, phenyl, tolyl, 2,4,6-trimethylphenyl, 1,2-isopropylmethylphenyl, 1-pentalenyl, naphthyl, anthryl. As used herein, the term “aralkyl” means an aromatic derivative of an alkyl group, preferably a (C2-C6)alkyl group, wherein the alkyl portion of the aromatic derivative may, optionally, be interrupted by an oxygen atom, such as, for example, phenylethyl, phenylpropyl, 2-(1-naphthyl)ethyl, preferably phenylpropyl, phenyoxypropyl, biphenyloxypropyl.

As used herein, viscosity may be evaluated using any suitable method. Unless indicated otherwise, viscosity is measured at 25° C. with a Brookfield (DV1) Viscometer.

Provided is a composition and process for producing a silicone-based material. The composition comprising a mixture of components for producing a silicone-based material, and comprises a siloxane comprising a hydrophilic group component. The use of the siloxane comprising a hydrophilic group has been found to provide a self-catalyzing activity such that an external catalyst is not needed to produce the silicone-based material. The process and composition can be utilized to provide a silicone-based material such as, for example, a silicone pressure sensitive adhesive. The resulting material produced from the composition and process may exhibit excellent properties and avoid issues surrounding the use of external catalysts including solution clarity, reduced viscosity build up, lower cyclic content, and others.

The composition for forming the silicone material includes (i) a silicone resin; (ii) a polyorganosiloxane, and (iii) siloxane comprising a hydrophilic group. The silicone material (iii) can be selected from a polyorganosiloxane and/or a siloxane comprising a hydrophilic group. The composition is substantially free of or completely free of any external catalyst. As used herein, the composition is considered substantially free of an external catalyst where there is less than 0.1 wt. %, less than 0.01 wt. %, or less than 0.001 wt. % of catalyst in the composition.

The composition includes a silicone resin (i). Silicone resins are typically referred to as MQ silicone resins or MQ resins comprising M units represented by the formula R³SiO_{1/2}, and Q units represented by the formula SiO_{4/2}, where R is generally selected from a C1 to C60 hydrocarbon. The hydrocarbon can be selected from an alkyl radical, a cycloaliphatic radical, an unsaturated hydrocarbon radical, and an aromatic radical. In one embodiment, R is selected from a C1-C60 alkyl radical, a C2-C60 unsaturated radical, a C5-C60 cycloaliphatic radical, and a C6-C60 aromatic radical. In one embodiment, R is selected from a C1-C10 alkyl radical. In one embodiment, R is selected from methyl, ethyl, propyl, isopropyl, butyl, isobutyl, tert-butyl, pentyl, hexyl, heptyl, or octyl.

In one embodiment, each R group is independently selected from a C1-C6 monovalent hydrocarbon, a C5-C20 cycloaliphatic radical, a C2-C6 olefinic radical, and a C6-C20 aromatic radical. Examples of suitable C1-C6 monovalent hydrocarbon radicals include, but are not limited to, methyl, ethyl, propyl, isopropyl, butyl, isobutyl, tert-butyl, pentyl, isopentyl, and hexyl. Examples of suitable cycloaliphatic radicals include, but are not limited to, cyclopyentyl, cyclohyexyl, cycloheptyl, cyclooctyl, etc. Examples of suitable C2-C6 olefinic radicals include, but are not limited to, vinyl, allyl, etc. Examples of suitable aromatic radicals include, but are not limited to, phenyl. The respective R groups can be the same or different within a given M, D, or T unit. In one embodiment, from about 95 to 100% of the R groups are methyl. In one embodiment, substantially all the R groups are free of unsaturation. In one embodiment, the MQ resin has from 0 to 0.5 mole % of the R groups contain any unsaturation.

The MQ resin is primarily formed of such M and Q units but may contain some residual D units (R₂SiO_{2/2}) and T units (RSiO_{3/2}). Generally, the MQ resin contains less than 20 mole % of D and T units, less than 15 mole % of D and T units, less than 10 mole % of D and T units, less than 5 mole % of D and T units, even less than 1 mole % of D and T units.

The silicone resin may have a ratio of M to Q units of from about 0.2:1 to about 1.7:1, from about 0.4:1 to about 1.5:1, from about 0.6:1 to about 1.2:1, or from about 0.8:1 to about 1:1.

The silicone resin may include some residual, free silanol (Si—OH) groups. In one embodiment, the silicone resin may have a silanol content of from about 0.5 wt. % to about 12.0 wt. %, from about 0.75 wt. % to about 10 wt. %, from about 1 wt. % to about 7.5 wt. %, or from about 2.5 wt. % to about 5 wt. % based on the weight of the silicone resin.

Silicone resin can also be a solid, a liquid, a dispersion, or solution in a solvent.

In embodiments, the silicone resin can have a viscosity of from about 1 cps to about 10000 cps, from about 10 cps to about 7500 cps, from about 25 cps to about 5000 cps, from about 50 cps to about 2500 cps, or from about 75 cps to about 1000 cps. In embodiments, the silicone resin can have a viscosity of from about 1 to about 100 cps, from about 100 to about 1000 cps, or from about 1000 to about 10000 cps.

The viscosity of the silicone resin can be based on the silicone resin as a liquid; or in a dispersion or solution, where the solids content of the silicone resin in the dispersion or solution is from about 10% solids to about 98% solids. In one embodiment, the viscosity is based on solution with about 60% solids. The silicone resin can be provided by a single type of silicone resin or a mixture of two or more different types of resin. The different resins can differ in terms of structure, viscosity, molecular weight, ratio of M to Q units and the like.

In accordance with aspects of the invention, the silicone resin is provided as a solid resin and is made by removing volatile solvent from the resin to provide a solid, solvent free MQ resin. This can be accomplished via spray drying.

The polyorganosiloxane (ii) is selected from a silanol terminate polyorganosiloxane. In one embodiment, the polyorganosiloxane (ii) is a silanol terminated compound having the formula:

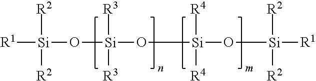

where R¹ is hydroxy (—OH) and R², R³, and R⁴ are independently selected from a C1-C60 hydrocarbon that may optionally be substituted with one or more halogen atoms, and n+m≥50 at 25° C.

In one embodiment, R², R³, and R⁴ are independently selected from a C1-C60 alkyl, a C1-C60 fluoroalkyl, a C2-C60 alkenyl, a C6-C60 aromatic containing group, phenyl, aryl, arylalkyl, fluoroalkyl groups, or a combination of two or more thereof. The aromatic containing compounds can include alkyaryl groups, arylalkyl groups, groups containing two or more aromatic rings, which can be separated by a bond, a linker group, or may be fused. In one embodiment, R², R³, and R⁴ are independently selected from a C1-C10 alkyl, a C6-30 aromatic containing, or a combination of two or more thereof. In one embodiment, R² is selected from a C1-C10 alkyl, R³ is selected from a C1-C10 alkyl, and R⁴ is selected from a C6-C30 aromatic group. In one embodiment, R², R³, and R⁴ are each methyl. In one embodiment, R² and R³ are methyl, and R⁴ is phenyl.

The polyorganosiloxane (ii) may have a viscosity of from 300 to 200,000,000, from about 500 to about 150,000,000, from about 1,000 to about 100,000,000, from about 2,500 to about 75,000,000, from about 5,000 to about 50,000,000, from about 10,000 to about 25,000,000, from about 20,000 to about 10,000,000, from about 30,000 to about 5,000,000, from about 50,000 to about 1,000,000, from about 75,000 to about 750,000, from about 100,000 to about 500,000, or from about 250,000 to about 400,000 centipoise (cps) at 25° C. and preferably The viscosity of polyorganosiloxane (ii) can be readily measured employing known and conventional viscosity measurement apparatus and techniques.

In one embodiment, the polyorganosiloxane (ii) has a viscosity of from about 50,000 to about 750,000, from about 75,000 to about 500,000, from about 100,000 to about 400,000, or from about 200,000 to about 300,000 cps.

It will be appreciated that the polyorganosiloxane (ii) can be provided as a mixture of two or more different polyorganosiloxane compounds. The different polyorganosiloxane can differ from one another in terms of structure, viscosity, size, and the like. In one embodiment, the polyorganosiloxane (ii) comprises a mixture of a first polyorganosiloxane having alkyl groups, and a second polyorganosiloxane comprising alkyl and aromatic groups. In one embodiment, the polyorganosiloxane (ii) comprises a first polyorganosiloxane having a first viscosity, and a second polyorganosiloxane having a second viscosity. In one embodiment, the first polyorganosiloxane has a viscosity of greater than 15,000 cPs, and the second polyorganosiloxane has a viscosity less than 12,000 cPs. In one embodiment the first polyorganosiloxane has a viscosity of from about 15,000 cPs to about 1,000,000 cPs, from about 25,000 cPs to about 750,000 cPs, from about 50,000 cPs to about 500,000 cPs, or from about 75,000 cPs to about 250,000 cPs; and the second polyorganosiloxane has a viscosity of from about 500 cPs to about 12,000 cPs, from about 1,000 cPs to about 10,000 cPs, from about 2,500 cPs to about 7,500 cPs, or from about 3,000 cPs to about 5,000 cPs.

The silicone resin (i) can be present in an amount of from about 40 wt. % to about 70 wt. %, from about 45 wt. % to about 65 wt. %, or from about 50 wt. % to about 55 wt. % based on the total weight of the silicone resin (i) and the polyorganosiloxane (ii). The polyorganosiloxane (ii) can be present in an amount of from about 30 wt. % to about 60 wt. %, from about 35 wt. % to about 55 wt. %, or from about 45 wt. % to about 50 wt. % based on the total weight of the silicone resin (i) and the polyorganosiloxane (ii).

The composition comprises a siloxane comprising a hydrophilic group (iii). The siloxane comprising a hydrophilic group can be selected from a hydrophilic functional silicone resin (e.g., a MQ type resin) and/or a hydrophilic functional polyorganosiloxane. The hydrophilic functional group is selected from an ionic group, an ionizable group, or a zwitterionic functional group.

In one embodiment, the siloxane comprising a hydrophilic group is a compound of the formula:

M¹ₐM²_{b}M³_{c}D¹_{d}D²ₑD³_{f}T¹_{g}T²ₕT³ᵢQ¹ⱼQ²ₖQ³ₗQ⁴ₒQ⁵ₚ

where

M¹=R⁵R⁶R⁷SiO_{1/2}

M²=R⁸R⁹R¹⁰SiO_{1/2}

M³=R¹¹R¹²R¹³SiO_{1/2}

D¹=R¹⁴R¹⁵SiO_{2/2}

D²=R¹⁶R¹⁷SiO_{2/2}

D³=R¹⁸R¹⁹SiO_{2/2}

T¹=R²⁰SiO_{3/2}

T²=R²¹SiO_{3/2}

T³=R²²SiO_{3/2}

Q¹=Si(OR²³)₁O_{3/2}

Q²=Si(OR²⁴)₂O_{2/2}

Q³=Si(OR²⁵)₃O_{1/2}

Q⁴=Si(OR²⁶)₄

Q⁵=SiO_{4/2}
where:
a, b, c, d, e, f, g, h, i, j, k, l, o, and p are zero or any positive integer subject to the following limitations:

$$1 \leq [ a + b + c + d + e + f + g + h + i + j + k + l + o + p ] \leq 6000 ;$$

$$[ b + e + h + k ] \geq 1 ;$$

R⁵, R⁶, R⁷, R⁹, R¹⁰, R¹², R¹³, R¹⁴, R¹⁵, R¹⁷, R¹⁹, and R²⁰ are each independently selected from hydrogen, a C1-C60 alkyl, a C6-C60 aromatic containing group, a C1-C10 alkoxy, and a hydroxyl;
R⁸, R¹⁶, R²¹, and R²⁴ are each independently selected from a monovalent hydrophilic group of the formula, where G≥0.

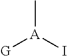

where A is a bridging unit between the siloxane moiety and the hydrophilic group I, having at least one spacing atom selected from a C1-C60 acyclic hydrocarbon radical, a C4-C60 alicylic radical, an C6-C60 aromatic hydrocarbon radical, a polyetheramine, a polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof;
I is an ionic, ionizable, zwitterionic group, or a polar hydrophilic group or moieties;
G is a polymerizable group having the general Formula:

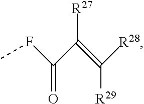

a non-polymerizable group of formula —F—K, wherein F is a linker group chosen from a C1-C60 acyclic hydrocarbon radical, a C4-C60 alicylic radical, an C6-C60 aromatic hydrocarbon radical, a polyetheramine, a polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof,
R²⁷, R²⁸, and R²⁹ are independently chosen from hydrogen or monovalent hydrocarbon radical with 1-5 carbon atoms, and
K is independently chosen from hydrogen, a C1-C60 acyclic hydrocarbon radical, a C4-C60 alicylic radical, an C6-C60 aromatic hydrocarbon radical, an epoxy, or an ether group;
R¹¹, R¹⁸, R²² are each independently selected from the group -A-G where A and G are as described above; and
R²³, R²⁵, and R²⁶ can be independently selected from R⁵ to R²² or a combination of thereof.

In one embodiment, A is a C1-C10 alkyl, a C1-C10 cycloalkyl, or a C6-C12 aromatic group. In one embodiment, A is a C1-C4 alkyl. It will be appreciated that the radical A will be divalent or trivalent depending on whether G is 0 or 1.

Where I is an ionic or ionizable group, I can be selected from an acid or base comprising a carboxylate —COO⁻, a dicarboxylate (—R(COO—)₂) a sulfone —SO₂—, a sulfonate —SO₃⁻, a sulfate —OSO₃⁻, a phosphonate —PO₃²⁻, a phosphate —OPO₃²⁻ group, —N⁺R³⁰R³¹H, —N⁺H₂R³², —N⁺H₃, or an ammonium salt, each containing a hydrogen or a cation independently selected from an alkali metal, an alkali earth metal, a transition metal, a quaternary ammonium group, and a phosphonium group, where R³⁰, R³¹, and R³² are independently selected from a C1-C30 hydrocarbon.

Where I is a zwitterionic moiety, the zwitterionic moiety is selected from a group comprising an anionic group and a cationic group in a covalently bonded compound and having a net neutral charge. In one embodiment, the zwitterionic group is selected from a group having the formula —R³³—N⁺(R³⁴)₂—R³⁵—I^{z} where R³³ is a divalent hydrocarbon group having from 1 to 20 carbon atoms, R³⁴ is a monovalent hydrocarbon group having from 1 to 20 carbon atoms, R³⁵ is a divalent hydrocarbon group having from 2 to 20 carbon atoms; and I^{z} is an ionic group selected from a carboxylate —COO⁻, a sulfone —SO₂—, a sulfonate —SO₃⁻, a sulfate —OSO₃⁻, a phosphonate —PO₃²⁻, and a phosphate —OPO₃²⁻ group.

In one embodiment, I is a polar group or moiety. The polar group or moiety refers to a group that is not ionizable or ionic in nature but contains heteroatoms that render the group polar. Without being bound to any particular theory, the polar groups may act as hydrogen bond acceptor, and those having hydrogen atoms directly bonded to a heteroatom may additionally act as hydrogen bond donors. Examples of suitable polar groups or moieties include, but are not limited to, a polyetheramine, a polyether, a polyamide, a polyester, a polyurethane, a polysulfone, and/or a polycarbonate.

In one embodiment, the hydrophilic group is selected from a polyetheramine. The polyetheramine can be selected from a compound containing an amine group and at least one polyalkylene oxide group. In one embodiment, the polyetheramine is selected from a compound of the formula:

R³⁶—(O—R³⁷—)_{q}—NH₂
where R³⁶ is selected from a C1-C60 hydrocarbon, R³⁷ is selected from a C1-C60 hydrocarbon, and q is ≥1. In one embodiment, R³⁶ and R³⁷ are independently selected from a C1-C60 alkyl, a C4-C30 cycloalkyl, and a C6-C30 aromatic group, where R³⁶ and R³⁷ can optionally include an amine functional group. In one embodiment, R³⁶ and R³⁷ are independently selected from a C1-C10 alkyl, a C4-C10 cycloalkyl, and a C6-C20 aromatic group, where R³⁶ and R³⁷ can optionally include an amine functional group. In one embodiment, q is 1 to about 200, about 4 to about 100, about 10 to about 50, or about 20 to about 40. In one embodiment, q is from 2 to about 40, about 4 to about 30, about 6 to about 25, about 8 to about 20, or about 10 to about 15.

In one embodiment, the polyetheramine is selected from a compound of the formula:

R³⁶—(O—CH₂CH₂)ₓ—(O—CCH₂CH₂CH₂)_{y}—(O CH₂CH₂CH(CH₃))_{z}—NH₂
where R³⁶ is as described above, and x, y, and z are independently 0 to about 300, where x+y+z is ≥2.

Some examples of suitable polyetheramines include, but are not limited to:

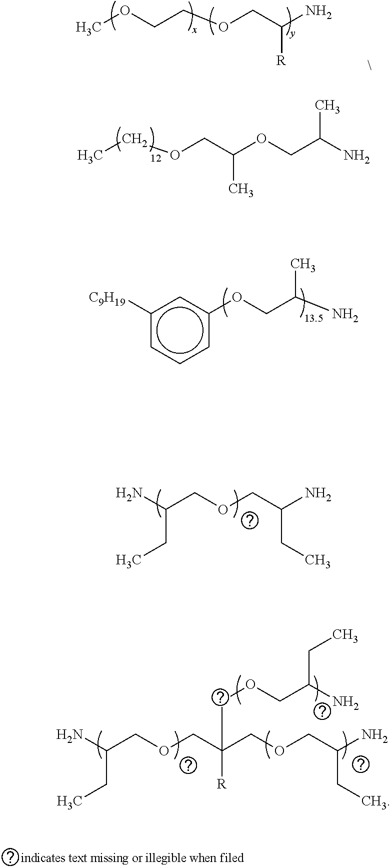

In one embodiment, the siloxane comprising a hydrophilic group (iii) is selected from a MD type polyorganosiloxane. In one embodiment, the hydrophilic functional siloxane (iii) is selected from a MQ type silicone resin comprising one or more M² groups with a hydrophilic functional group as described above. In an embodiment where the hydrophilic siloxane (iii) is a MQ type resin, the hydrophilic siloxane can be of the formula M¹ₐM²_{b}M³_{c}Q⁵ₚ, where b is ≥1.

The siloxane comprising a hydrophilic group (iii) can be a solid, a liquid, a dispersion, or solution in a solvent.

The siloxane comprising a hydrophilic group (iii), in embodiments, can have a viscosity of from 1 cps to about 10000 cps, from about 10 cps to about 7500 cps, from about 25 cps to about 5000 cps, from about 50 cps to about 2500 cps, or from about 75 cps to about 1000 cps. The siloxane comprising a hydrophilic group (iii), in embodiments, can have a viscosity of from about 1 cPs to about 100 cPs, from about 100 cPs to about 1000 cPs, or from about 1000 cPs to about 10000 cPs. The viscosity of the siloxane comprising a hydrophilic group can be based on the siloxane as a liquid; or in a dispersion or in solution, where the solids content of the siloxane comprising a hydrophilic group in the dispersion or solution is from about 10% solids to about 98% solids. In one embodiment, the viscosity is based on siloxane as a liquid.

The siloxane comprising a hydrophilic group (iii) can be present in an amount of from about 0.1 wt. % to about 20 wt. %, from about 0.5 wt. % to about 15 wt. % from about 1 wt. % to about 10 wt. %, from about 2 wt. % to about 8 wt. %, or from about 3 wt. % to about 5 wt. % based on the total weight of the composition.

The siloxane comprising a hydrophilic group are prepared by the reaction of an olefin bearing at least one polar group, and a silyl hydride in presence of a hydrosilylation catalyst.

The silyl hydride is chosen from a silicon containing compound comprising at least one —SiH group. The —SiH group can be part of a M, D, or T unit of the siloxane. In one embodiment, a silyl hydride is selected where —SiH is part of M units bonded to Q unit. As used herein, M represents a monofunctional group of formula R3SiO1/2, D represents a difunctional group of formula R2SiO2/2, T represents a trifunctional group of formula RSiO3/2, Q represents a tetrafunctional group of formula SiO4/2.

Some non-limiting examples of silyl hydrides include, Pentamethyldisiloxane such as P1535, tetramethyldisiloxane such as T1437, heptamethyltrisiloxane such as H1267, Tris(trimethylsiloxy)silane such as T3520 available from TCI; polymethylhydrosiloxane include HMS-082, HMS 501 HPM-502, 65 HMS-992, HMS-064, available from Gelest; polyhydrosilsesquioxane, octakis(dimethylsiloxy)-T8-silsesquioxane such as SI06696.5 from Gelest, and other hydride-containing copolymers or homopolymers of dimethyl siloxane or phenyl-containing siloxanes such as HDP-111-hydride terminated poly-15 phenyl(dimethylhydrosiloxy)siloxane, available from Gelest.

Other examples of silyl hydride agents include, but are not limited to, a Q resin, which may also be referred to as MQ hydride or hydride modified silica Q resins. These silyl hydrides have an activity of 1-25 equivalents/kg. Examples of those compounds include, but are not limited to, those commercially available under the tradename MQH-9 (Milliken & Company), which is a hydride-modified silica Q resin characterized by a molecular weight of 900 g/mole and an activity of 9.5 equivalents/kg; HQM 105 (Gelest, Inc.), which is a hydride modified silica Q resin characterized by a molecular weight of 500 g/mole and an activity of 8-9 equivalents/kg; and HQM 107 (Gelest, Inc), which is a hydride-modified silica Q resin characterized by a molecular weight of 900 g/mole and an activity of 8-9 equivalents/kg. The solids content of the resulting pressure sensitive adhesive can be adjusted as desired with an appropriate solvent. The solids content of the pressure sensitive adhesive can be selected as desired for a particular purpose or intended application. In one embodiment, the solids content of the pressure sensitive can be adjusted to be from about 30% to about 80%, from about 40% to about 70%, or from about 50% to about 60%. The solvent is preferably a non-aromatic solvent and, more preferably a solvent other than a BTX (benzene, toluene, xylene) type solvent. Examples of suitable solvents that may be used to dissolve the pressure sensitive adhesive include, but are not limited to, hydrocarbon solvents, silicone solvents, an amide, an ester, a ketone, an alcohol, or an ether. The solvent can be a natural or synthetic material.

Examples of suitable aliphatic hydrocarbons include linear, branched, or cyclic aliphatic hydrocarbons having 6 to 16 carbon atoms, for example saturated acyclic aliphatic hydrocarbons (paraffins) such as heptane, hexane, octane, isooctane, decane, dodecane, and their isomers such as, but not limited to, isodecane, isohexadecane or isododecane and cyclic aliphatic hydrocarbons such as cyclohexane, methylcyclohexane or decahydronaphthalene. The aliphatic hydrocarbon solvent can be an alkene, for example heptene, cyclohexadiene, cyclohexene, or 2,5-dimethyl-2,4-hexadiene. Mixtures of aliphatic hydrocarbons are also suitable, for example the mixture of branched paraffins sold under the trademark ISOPAR®. Other suitable materials include natural terpenes such as, but not limited to pinene isomers, myrcene, bisabolnee, cadinene, and the like.

Examples of suitable volatile silicone solvents include, but are not limited to, linear, branched, and cyclic polydiorganosiloxanes, for example polydimethylsiloxanes such as linear trimethylsilyl-terminated polydimethylsiloxanes having a viscosity of 0.65 to 5 cP at 25° C., and cyclic polydimethylsiloxanes such as decamethylcyclopentasiloxane and octamethylcyclotetrasiloxane. Volatile silicone solvents can contain organic groups other than methyl, for example higher alkyl groups or phenyl groups. An example is 3-octyl heptamethyl trisiloxane. In one embodiment, viscosity is determined by dissolving the melt obtained PSA into a suitable solvent at 60% solids and measuring the viscosity at 25° C. with a Brookfield (DV1) Viscometer.

Examples of suitable ester solvents include, but are not limited to, carboxylate esters such as alkyl carboxylate esters and carbonate esters such as alkyl carbonate esters. For example the volatile solvent can comprise at least one C1-C8 alkyl ester of a C2-C4 carboxylic acid such as ethyl acetate or butyl acetate. Examples of suitable carbonate ester solvents include, but are not limited to, diethyl carbonate and dicaprylyl carbonate.

Examples of suitable ketone solvents include, but are not limited to, methyl isobutyl ketone (4-methyl-2-pentanone), 2-pentanone, 3-hexanone, methyl isoamyl ketone (5-methyl-2-hexanone), camphor, menthone, carvone, pulegone, and the like.

Examples of suitable ether solvents include, but are not limited to, dibutyl ether, volatile polyethers such as 1-(propoxymethoxy)propane and cyclic ethers such as cyclopentamethyl ether.

Examples of suitable alcohol solvents include, but are not limited to methanol, ethanol, propanol, butanol, cis-3-hexanol, trans-2,cis-6-nonadienol cis-6-noneol, linalool, geraniol, nerol, citronellol, nerolidol, farnesol, benzyl alcohol, phenylethyl alcohol, cinnamic alcohol, citronellol, hydroxycitronellal, linalool, dihydrolinalool, tetrahydrolinalool, ethyllinalool, geraniol, nerol, tetrahydrogeraniol, myrcenol, dihydromyrcenol, tetrahydromyrcenol, ocimenol, terpineol, menthol, borneol, fenchyl alcohol, farnesol, nerolidol, cedrol, and terpineol and the like.

The silicone pressure sensitive adhesives prepared by the method of this invention will readily stick to support a solid support or substrate, whether flexible or rigid. These pressure sensitive adhesive compositions may be applied to a surface by any suitable means such as rolling, spreading or spraying. The surface of the support and the substrate to which the support is adhered may be any known solid material such as metals, paper, wood, leather, fabrics, organic polymeric materials, painted surfaces, siliceous materials such as concrete, bricks, cinderblocks, and glass including glass cloth. After applying it to the surface, the adhesive may be cured by air drying or heating for example at temperatures of up to 300° C.

Additionally, the pressure sensitive adhesives produced by the present technology may exhibit a low concentration of cyclic siloxanes. In embodiments, the pressure sensitive adhesive contains less than about 2500 ppm, less than 2000 ppm, less than 1800 ppm, less than 1500 ppm, less than 1250 ppm, less than 1000 ppm less than 750 ppm, less than 500 ppm, less than 250 ppm, even less than 100 ppm of one or more of a octamethylcyclotetrasiloxane (D4), decamethylcyclopentasiloxane (D5), or dodecamethylcyclohexasiloxane (D6). In one embodiment, the pressure sensitive adhesive contains each of a D4, D5, or D6 cyclic siloxane in an amount of less than 2500 ppm, less than 2000 ppm, less than 1800 ppm, less than 1500 ppm, less than 1250 ppm, less than 1000 ppm less than 750 ppm, less than 500 ppm, less than 250 ppm, even less than 100 ppm.

An adhesive material formed from the present compositions can be provided by reacting the composition in the presence of heat for a sufficient time to effect condensation of the silicone resin and the silicone polymer to increase molecular weight or crosslinking, or both. The process comprises reacting a polyorganosiloxane and a silicone resin in the presence of a hydrophilic functional siloxane (e.g., a hydrophilic functionalized polyorganosiloxane and/or hydrophilic functionalized silicone resin). In one embodiment, a solid silicone resin (in the form of a MQ type resin) is dispersed in a polyorganosiloxane polymer and heated at a temperature of from about 80 to about 150° C. The hydrophilic functionalized polyorganosiloxane and/or hydrophilic functionalized silicone resin are added to the mixture of the polyorganosiloxane and the silicone resin and heated for a period of time sufficient for a condensation reaction to occur to a selected extent. In one embodiment, no additional catalyst (e.g., metal catalysts such as, but not limited to, platinum based catalysts) is required to form the cured material.

A pressure sensitive adhesive formed from the present compositions is suitable for a number of applications. The pressure sensitive adhesives exhibit suitable hydrophilicity while also exhibiting excellent adhesion and other properties desirable for a number of different applications. In one embodiment, the pressure sensitive adhesives can be employed in the fields of applications such as gels, emulsions, healthcare applications, household applications, apparel, sporting goods such as diving masks, paints, coatings, fuel cell, electronic and electro-optic applications, agriculture, membranes, injection moldable and compression moldable rubbers and plastics, and various silicone based rubbers, production of domestic appliances, machine and instrument construction, coatings such as release coatings, protective coatings, antifouling coatings, and consumer goods such as, but not limited to, personal care products, beauty products, cosmetic products and the like. As a specific example, in the healthcare field, silicone elastomers are used for the fabrication of wound dressings, dressings for scar reduction, drug delivery devices, medical tubing, clinical surfaces, pacemaker leads, pressure sensitive adhesives, wound healing patches, wound management device, medical adhesives, catheters, shunts, valves, stents, transdermal patches, scaffold for tissue engineering, anti-microbial devices, ophthalmic devices, bioinserts, plugs, surgical devices, medical devices, devices for medical storage, childcare products, assisted breathing apparatus, prostheses, reconstructive devices, and body implants.

The adhesives may find use in personal care products. The adhesives may find utility to adhere functional articles which adhere to the skin such as cosmetic or pharmaceutical delivery articles which provide a substance to the skin such as skin treatment substances, cream, lotions, hormones, vitamins, deodorants, drugs; cosmetic or pharmaceutical delivery articles provide a substance to emanate away from the skin such as insecticides, inhalation drugs, perfumes, etc.; functional articles which are not necessarily attached to the skin, but which require a high residence time on the skin such as decorative cosmetics, lipstick, eye shadow, stage make-up) and cleaning articles (hand cleaners, face masks and hygienic pore cleansers). The adhesive may also in addition find application to attach articles to the skin such as protective articles such as knee-, or elbow-protectors or bandages; clothing such as bras, surgical gowns, or parts of garments during fitting at a tailor; nasal plasters; prosthesis such as breast replacements or wigs; cold wraps e.g. to provide pain relief from bruises and to reduce swelling; thermal wraps to provide relief of temporary and chronic pain such as neck wraps, knee wraps; hearing aids; protective face masks (for the reduction or prevention of inhalation of noxious substances); anti-snoring patches, ornamental articles such as jewelry, earrings, guises, tattoos; goggles or other eye wear, tapes, bandages, and the like.

The curable compositions of the present invention can be rendered antimicrobial via contact with an antimicrobial agent exemplified by but not limited to silver, copper, zinc, chlorhexidine, benzalkonium chloride, biguanide, polyquaternary ammonium compounds, polyquaternary phosphonium compounds, chitosan and its derivatives, antimicrobial peptides such as but not limited to nisin, pediocin, gomesin, pleuricidin and their derivatives and their recombinant forms which can bind to the ionic groups present on the silicone ionomers of the present invention.

Such microbiocides can be released upon contact with physiological or clinical environments and create a transient or permanent antimicrobial effect at the contact site.

In yet another example, addition cured pressure sensitive silicone adhesives are used for the attachment of wound dressings to the periwound skin. Silicones do not stick to the wound bed, but adhere to the skin. Hence they can be removed or repositioned atraumatically.

The present technology has been described in the foregoing detailed description and with reference to various aspects and embodiments. The technology may be further understood with reference to the following Examples. The Examples are intended to further illustrate aspects and embodiments of the present technology and not necessarily to be limited to such aspects or embodiments.

### EXAMPLES

### MQ Silicone Resin

The solid MQ silicone resin used to prepare PSA. The solid MQ silicone resins which were used to prepare the pressure sensitive adhesives have a silanol content of 95473 ppm to 47746 ppm. The MQ resins employed are referred as MQ1-(silanol content of 95473 ppm), MQ2 (silanol content of 47746 ppm), MQ3 (silanol content of 56642 ppm), and MQ4 (silanol content of 80722 ppm) resin.

### Polyorganosiloxane

### Component 1-3

Components 1, 2, and 3 illustrate the polyorganosiloxane in accordance with aspects and embodiments of the present methods and their properties like molecular weight (MW) and cyclic content.

MW of components, examples and comparative examples was determined by Gel Permeation Chromatography (GPC) using Chloroform solvent and calibrated using polystyrene standards. The viscosity of components, examples and comparative examples was determined at 25° C. with a Brookfield (DV1) Viscometer using spindle #2 to #6. Cyclic content of components, examples and comparative examples was quantified using gas chromatographic method.

Polyorganosiloxane of Component 1 to 3 is listed in Table 1 below:

TABLE 1
Polyorganosiloxane

| | | | | | Octamethyl |
|---|---|---|---|---|---|
| | | | | | cyclotetrasiloxanes |
| | | | Viscosity | MW | (D4) |
| Component | Description | CAS# | (cPs) | (g/mol) | Wt. % |
| 1 | Polyorganosiloxane | 556-67-2 | 50,000-250,000 | 2.34 × 10⁵ | <10 |
| 2 | Polyorganosiloxane | 556-67-2 | 2550-3570 | 3.74 × 10⁴ | <1 |
| 3 | Polyorganosiloxane | 556-67-2 | 300,000-750,000 | 5.8 × 10⁵ | <10 |

### Siloxane Comprising a Hydrophilic Group Component 4

A dicarboxylate polydimethylsiloxane was prepared as described in synthetic example 1 of EP3280496A1

### Component 5

Silsoft™ BAPD fluid, an amino polydimethylsiloxane was used as component 5.

### Component 6

MQH-9, a MQ hydride (100 grams) was taken in a 500 mL three round bottom flask fitted with water condenser, thermopocket, and dropping funnel under N₂ sparging. The dropping funnel was filled with allyl succinic anhydride (CAS No. 7539-12-0) (72.2 g). The content of the round bottom flask was heated to 85° C., and 7.22 g of allyl succinic anhydride was charged in the round bottom flask. To this mixture, 10 ppm of Karstedt's catalyst (CAS No. 68478-92-2) was added followed by dropwise addition of the remaining allyl succinic anhydride in the dropping funnel. The reaction was allowed to proceed for 4 to 5 hours. Triisopropoxy(vinyl)silane (CAS No. 18023-33-1) (119.8 g) was then added to the dropping funnel and added to the round bottom flask dropwise, and the reaction was allowed to again proceed for 4 to 5 hours. After the reaction, the round bottom flask was cooled to 50° C. and was maintained under vacuum for 2 hours to obtain ionic MQ resin. Ionic MQ resin was analyzed using GPC having a unimodal resin peak.

### Component 7

MQH-9, a MQ hydride (100 grams) was taken in a 500 mL three round bottom flask fitted with water condenser, thermopocket, and dropping funnel under N₂ sparging. The dropping funnel was filled with vinylcyclohexene oxide (CAS No. 106-86-5) (64.0 g). The content of round bottom flask was heated to 85° C., and 6.4 g of vinylcyclohexene oxide was charged in the round bottom flask. To this mixture, 10 ppm of Karstedt's catalyst (CAS No. 68478-92-2) was added followed by dropwise addition of the remaining vinylcyclohexene in the dropping funnel. The reaction was allowed to proceed for 4 to 5 hours. Triisopropoxy(vinyl)silane (CAS No. 18023-33-1) (119.8 g) was then added to the dropping funnel and added to the round bottom flask dropwise, and reaction was allowed to again proceed for 4 to 5 hours. After the reaction, the round bottom flask was cooled to 50° C. and Jeffamine M-600 (CAS No. 83713-01-3) (300.93 g) was addend and reaction was allowed to proceed for 24 hours. After 24 hours contents of the round bottom flaks were maintained under vacuum for 2 hours to obtain polyether functional MQ resin. Polyether functional MQ resin was analyzed using GPC having a unimodal resin peak.

### Component 8

Ethylene Glycol Monoallyl Ether (CAS No. 111-45-5) (100 g), N,N-Dimethylcarbamyl chloride (CAS No. 79-44-7) (115.83 g), and 4-(Dimethylamino)pyridine (CAS No. 1122-58-3) (5.98 g) were taken in a 500 mL two neck round bottom flask fitted with a co ndenser and a thermopocket. The content of the round bottom flask were heated to 80° C. and the reaction was allowed to proceed for 24 hours to synthesize 2-allyloxyethyl N,N-dimethylcarbamate. The product was dissolved in hexane and washed with 1N HCl solution followed by 1 N sodium bicarbonate solution and brine. The organic layer was dried over anhydrous sodium sulfate, followed by removal of hexane using low pressure evaporation. 200 g of 2-allyloxyethyl N,N-dimethylcarbamate was obtained.

MQH-9, a MQ hydride (100 grams) was taken in a 1000 mL three round bottom flask fitted with water condenser, thermopocket, and dropping funnel under N₂ sparging. The dropping funnel was filled with 2-allyloxyethyl N,N-dimethylcarbamate (89.3 g). The content of the round bottom flask was heated to 85° C., and 8.9 g of 2-allyloxyethyl N,N-dimethylcarbamate was charged in the round bottom flask. To this mixture, 10 ppm of Karstedt's catalyst (CAS No. 68478-92-2) was added followed by dropwise addition of the remaining 2-allyloxyethyl N,N-dimethylcarbamate in the dropping funnel. The reaction was allowed to proceed for 4 to 5 hours. Triisopropoxy(vinyl)silane (CAS No. 18023-33-1) (119.8 g) was then added to the dropping funnel and added to the round bottom flask dropwise, and the reaction was allowed to again proceed for 4 to 5 hours. After the reaction, the round bottom flask was cooled to 50° C. Tetrahydrofuran (CAS No. 109-99-9) (300 mL) and chloroacetic acid (CAS No. 79-11-8) (48.7 g) were added to the round bottom flask and reaction was allowed to proceed for 24 hours. Tetrahydrofuran was removed under low pressure evaporation to obtain a zwitterionic MQ resin. The zwitterionic MQ resin was analyzed using GPC having a unimodal resin peak.

### Preparation of Silicone PSA

### Example 1

Polyorganosiloxane of component 1 (52.07 grams) and component 2 (52.07 grams) were added followed by MQ3 (171.84 grams) or vice versa in a 3-liter planetary mixture equipped with helical blade & heating apparatus, thermocouple, sparge tube (for N₂ sparging). The reactor temperature was initially set to 125-130° C. under positive nitrogen flow. The above mixture was finally agitated at 135-145° C. until a completely homogeneous solution/dispersion was obtained. The mixing process was continued for 1-4 hours until MQ3 was dissolved or dispersed completely in the polyorganosiloxane mixture. 12 grams (4%) of component 4 was added and the reaction was then continued for another 7 hours. The reactor temperature was maintained at 145° C. under vacuum for 1 to 2 hours. After the cooking step, the reactor was cooled. The solids content was then adjusted to 60% by dissolving the resulting high viscous mass in 40 parts ethyl acetate (˜200 grams) at 50° C. The GPC of the PSA had multimodal resin and polymer peaks.

### Example 2

Polyorganosiloxane of component 2 (18 grams) and component 3 (18 grams) were added followed by MQ4 (54 grams) in heptane (36 grams) was added in a glass reactor equipped with a helical blade, heating apparatus, thermocouple, and sparge tube (for N₂ sparging) and a Dean Stark water trap filled with heptane. The mixture was homogeneously mixed at 90° C. for 3 hours under positive nitrogen flow. 3.6 grams (4%) of component 4 was added and reaction continued for 12 hours. The temperature of the reaction was increased to 110° C. and the mixture refluxed until the last trace of water was observed. The PSA obtained was obtained in heptane solution. The GPC of the PSA had multimodal resin and polymer peaks.

### Example 3

Polyorganosiloxane of component 2 (36 grams) was added followed by MQ4 (54 grams) or vice versa in a 3-liter planetary mixture equipped with helical blade & heating apparatus, thermocouple, sparge tube (for N₂ sparging). The reactor temperature was initially set to 125-130° C. under positive nitrogen flow. The above mixture was finally agitated at 135-145° C. until a completely homogeneous solution/dispersion was obtained. The mixing process was continued for 1-4 hours until MQ4 was dissolved or dispersed completely in the polyorganosiloxane. 3.6 grams (4%) of component 5 was added and reaction continued for another 12 hours. After the cooking step, the reactor was cooled. The solids content was then adjusted to 60% by dissolving the resulting high viscous mass in 40 parts ethyl acetate (˜60 grams) at 50° C. The GPC of the PSA had multimodal resin and polymer peaks.

### Example 4

Polyorganosiloxane of component 1 (16.98 grams) and component 2 (16.98 grams) were added followed by MQ4 (53.79 grams) or vice versa in a grass reactor equipped with helical blade & heating apparatus, thermocouple, sparge tube (for N₂ sparging). The reactor temperature was initially set to 125-130° C. under positive nitrogen flow. The mixing process was continued overnight until MQ4 was dissolved or dispersed completely in the polyorganosiloxane mixture. 2.25 grams (2.5%) component 6 was added, and reaction was continued for another 12 hours. After the cooking step, the reactor was cooled. The solids content was then adjusted to 60% by dissolving the resulting high viscous mass in 40 parts ethyl acetate (˜60 grams) at 50° C. The GPC of the PSA had multimodal resin and polymer peaks.

### Example 5

Polyorganosiloxane of component 1 (16.98 grams) and component 2 (16.98 grams) were added followed by MQ4 (51.54 grams) or vice versa in a grass reactor equipped with helical blade & heating apparatus, thermocouple, sparge tube (for N₂ sparging). The reactor temperature was initially set to 125-130° C. under positive nitrogen flow. The mixing process was continued overnight until MQ4 was dissolved or dispersed completely in the polyorganosiloxane mixture. 4.5 grams (5%) component 6 was added, and reaction was continued for another 12 hours. After the cooking step, the reactor was cooled. The solids content was then adjusted to 60% by dissolving the resulting high viscous mass in 40 parts ethyl acetate (˜60 grams) at 50° C. The GPC of the PSA had multimodal resin and polymer peaks.

### Example 6

Polyorganosiloxane of component 2 (38.30 grams) was added followed by MQ4 (49.45 grams) or vice versa in a grass reactor equipped with helical blade & heating apparatus, thermocouple, sparge tube (for N₂ sparging). The reactor temperature was set to 125-130° C. under positive nitrogen flow. The mixing process was continued overnight until MQ4 was dissolved or dispersed completely in the polyorganosiloxane. 2.25 grams (2.5%) component 7 was added, and reaction was continued for another 7 hours. After the cooking step, the reactor was cooled. The solids content was then adjusted to 60% by dissolving the resulting high viscous mass in 40 parts ethyl acetate (˜60 grams) at 50° C. The GPC of the PSA had multimodal resin and polymer peaks.

### Example 7

Polyorganosiloxane of component 2 (38.30 grams) was added followed by MQ4 (47.20 grams) or vice versa in a grass reactor equipped with helical blade & heating apparatus, thermocouple, sparge tube (for N₂ sparging). The reactor temperature was set to 125-130° C. under positive nitrogen flow. The mixing process was continued overnight until MQ4 was dissolved or dispersed completely in the polyorganosiloxane. 4.5 grams (5%) component 7 was added, and reaction was continued for another 7 hours. After the cooking step, the reactor was cooled. The solids content was then adjusted to 60% by dissolving the resulting high viscous mass in 40 parts ethyl acetate (˜60 grams) at 50° C. The GPC of the PSA had multimodal resin and polymer peaks.

### Example 8

Polyorganosiloxane of component 2 (38.30 grams) was added followed by MQ4 (42.70 grams) or vice versa in a grass reactor equipped with helical blade & heating apparatus, thermocouple, sparge tube (for N₂ sparging). The reactor temperature was set to 125-130° C. under positive nitrogen flow. The mixing process was continued overnight until MQ4 was dissolved or dispersed completely in the polyorganosiloxane. 9.0 grams (10%) component 7 was added, and reaction was continued for another 7 hours. After the cooking step, the reactor was cooled. The solids content was then adjusted to 60% by dissolving the resulting high viscous mass in 40 parts ethyl acetate (˜60 grams) at 50° C. The GPC of the PSA had multimodal resin and polymer peaks.

### Example 9

Polyorganosiloxane of component 1 (16.98 grams) and component 2 (16.98 grams) were added followed by MQ4 (53.79 grams) or vice versa in a grass reactor equipped with helical blade & heating apparatus, thermocouple, sparge tube (for N₂ sparging). The reactor temperature was set to 125-130° C. under positive nitrogen flow. The mixing process was continued overnight until MQ4 was dissolved or dispersed completely in the polyorganosiloxane. 2.25 grams (2.5%) component 8 was added, and reaction was continued for another 7 hours. After the cooking step, the reactor was cooled. The solids content was then adjusted to 60% by dissolving the resulting high viscous mass in 40 parts ethyl acetate (˜60 grams) at 50° C. The GPC of the PSA had multimodal resin and polymer peaks.

### Example 10

Polyorganosiloxane of component 1 (16.98 grams) and component 2 (16.98 grams) were added followed by MQ4 (51.54 grams) or vice versa in a grass reactor equipped with helical blade & heating apparatus, thermocouple, sparge tube (for N₂ sparging). The reactor temperature was set to 125-130° C. under positive nitrogen flow. The mixing process was continued overnight until MQ4 was dissolved or dispersed completely in the polyorganosiloxane mixture. 4.5 grams (5%) component 8 was added, and reaction was continued for another 7 hours. After the cooking step, the reactor was cooled. The solids content was then adjusted to 60% by dissolving the resulting high viscous mass in 40 parts ethyl acetate (˜60 grams) at 50° C. The GPC of the PSA had multimodal resin and polymer peaks.

### Example 11

Polyorganosiloxane of component 1 (16.98 grams) and component 2 (16.98 grams) were added followed by MQ4 (47.04 grams) or vice versa in a grass reactor equipped with helical blade & heating apparatus, thermocouple, sparge tube (for N₂ sparging). The reactor temperature was set to 125-130° C. under positive nitrogen flow. The mixing process was continued overnight until MQ4 was dissolved or dispersed completely in the polyorganosiloxane mixture. 9.0 grams (10%) component 8 was added, and reaction was continued for another 7 hours. After the cooking step, the reactor was cooled. The solids content was then adjusted to 60% by dissolving the resulting high viscous mass in 40 parts ethyl acetate (˜60 grams) at 50° C. The GPC of the PSA had multimodal resin and polymer peaks.

### Comparative Example 1

Polyorganosiloxane of component 2 (153.19 grams) was added followed by MQ4 (206.81 grams) or vice versa in a 3-liter planetary mixture equipped with helical blade & heating apparatus, thermocouple, sparge tube (for N₂ sparging). The reactor temperature was set to 125-130° C. under positive nitrogen flow. The above mixture was agitated at 125-130° C. until a completely homogeneous solution/dispersion was obtained. The mixing process was continued for 1-4 hours until MQ4 was dissolved or dispersed completely in the polyorganosiloxane. 2.0 grams of 2,4,6-Trimethyl-2,4,6-trivinylcyclotrisilazane (CAS-No:5505-72-6) was added and reaction continued for another 3 hours. Finally, the reactor temperature was increased to 150° C. and hold the temperature for 2-3 hours under N₂. After the cooking step, the reactor was cooled. The solids content was then adjusted to 6000 by dissolving the resulting high viscous mass in 40 parts ethyl acetate (˜200 grams) at 50° C. The GPC of the PSA had multimodal resin and polymer peaks.

TABLE 2
Summary of PSA Examples

| | | | Siloxane | Functional | |
|---|---|---|---|---|---|
| | MQ | | comprising a | Component | |
| | Silicone | | hydrophilic | concentration | R/G |
| PSA | Resin | Polyorganosiloxane | group | (wt %) | ratio |
| Example 1 | MQ3 | Component 1 and | Component 4 | 4% | 1.65 |
| | | Component 2 | | | |
| Example 2 | MQ4 | Component 1 and | Component 4 | 4% | 1.5 |
| | | Component 2 | | | |
| Example 3 | MQ4 | Component 2 | Component 5 | 4% | 1.5 |
| Example 4 | MQ4 | Component 1 and | Component 6 | 2.5% | 1.65 |
| | | Component 2 | | | |
| Example 5 | MQ4 | Component 2 and | Component 6 | 5% | 1.65 |
| | | Component 3 | | | |
| Example 6 | MQ4 | Component 2 | Component 7 | 2.5% | 1.35 |
| Example 7 | MQ4 | Component 2 | Component 7 | 5% | 1.35 |
| Example 8 | MQ4 | Component 2 | Component 7 | 10% | 1.35 |
| Example 9 | MQ4 | Component 2 and | Component 8 | 2.5% | 1.65 |
| | | Component 3 | | | |
| Example 10 | MQ4 | Component 1 and | Component 8 | 5% | 1.65 |
| | | Component 2 | | | |
| Example 11 | MQ4 | Component 2 | Component 8 | 10% | 1.65 |
| Comparative | MQ4 | Component 2 | NA | 0% | 1.35 |
| Example 1 | | | | | |

### Adhesive Performance

PSA sample was coated to a thickness of 70-80 micron for probe tack and peel strength adhesion measurements. The adhesives were coated on 3M Scotchpak™ 9733 backing polyester film laminate and using 3M Scotchpak™ 1022 release liner. Adhesives were dried for 10 minutes at 90° C. in oven. Tack testing was done as per FINAT FTM 9 for measuring tack with units of grams. Peel adhesion testing was done per ASTM D3330/D3330M measuring adhesion to mirrored stainless steel plates at 180′ peel angle. Peel adhesion results were recorded for 1-inch strips as N/in peel force from stainless steel panels at 12 inch per minute peel speed.

TABLE 3
Adhesive performance

| | Probe Tack | Peel strength |
|---|---|---|
| PSA | Adhesive Force (N) | Adhesive strength (N/mm) |
| Example 5 | 6.12 | 7.50 |
| Example 6 | 5.89 | 10.77 |
| Example 7 | 6.65 | 5.77 |
| Example 8 | 3.98 | 6.28 |
| Example 9 | 2.69 | 8.20 |
| Example 10 | 4.34 | 12.50 |
| Comparative | 2.52 | 18.90 |
| Example 1 | | |

### Cyclic Siloxane Estimation

Cyclic siloxane present in PSA were quantified using gas chromatographic method. Sample was extracted for 24 hours as described below.

### Experimental Method:

Approximately 0.5 g sample was weighed in 20 mL vial. Accurate weight was noted down. Sample was extracted for 24 hours in 10 mL working solution prepared using acetone as solvent and toluene and dodecane as internal standards (0.05 mg/mL). Calibration plots for cyclic siloxanes D4, D5 and D6 were generated by preparing various standards of concentration varying from 0.005 to 0.1 mg/mL in acetone. Peak area was normalized by dividing with peak area for dodecane and plotted against concentration. Cyclic siloxanes present in the sample were calculated using normalized peak area in GC chromatogram of sample and the calibration curve.

### GC Conditions

Instrument: Agilent 7890A GC
Detector: FID at 350° C.; H2: 30 mL/min, Air: 300 mL/min, makeup flow: 25.4
mL/min
Sample Introduction: Split Injection/225° C./50:1 split ratio
Injection Volume: 2 μL (Agilent 7693 Autosampler)
Carrier Gas: Nitrogen (1.4 mL/min, constant flow mode)
Capillary Column: Agilent HP-5 (30 m×0.32 mm×0.25 m)
Temperature Program: 50° C. (5 min) to 200° C. at 15° C./min then to 315° C. (20 min)

TABLE 4
Cyclic siloxanes estimation

| | Cyclic siloxane content (ppm) | | |
|---|---|---|---|
| PSA | D4 | D5 | D6 |
| Example 1 | <100 | <100 | <100 |
| Example 2 | 2589 | 2338 | 1153 |
| Example 3 | <50 | <100 | <100 |
| Example 4 | ND | ND | <100 |
| Example 5 | 310 | 207 | <100 |
| Example 6 | ND | <100 | <100 |
| Example 7 | ND | 475 | ND |
| Example 8 | ND | 157 | <100 |
| Example 9 | ND | <100 | 115 |
| Example 10 | ND | ND | <100 |
| Example 11 | 835 | 925 | 498 |
| Comparative Example 1 | <50 | 110 | <100 |

*ND: Not detected

### Viscoelastic Properties

The viscoelastic properties of the silicone PSA made by the described process herein, has been thoroughly characterized by the dynamic rheological analysis. The G′ at lower angular frequency (ω=0.01 rad/s) represents the adhesive strength during application of the adhesive whereas the G′ at higher angular frequency (ω=100 rad/s) relates to the peel force at debonding. The complex viscosity values on the other hand compares the cold flow properties of these adhesives. The data presented in the table-5 suggests that for all the given compositions [example-1 to 11], the PSA properties are comparable to those where the known state of the art condensation catalyst were used. The experimental process involves casting of PSA onto a suitable release liner and drying at 150° C. for 1 hour and then transferring from release linear to the DHR3 rheometer (TA Instruments. A 25 mm parallel plate geometry were used in oscillation mode with an angular strain of 0.01% to generate the rheograms.

TABLE 4
Rheology

| | | | G′ | G″ | | G′ | G″ |
|---|---|---|---|---|---|---|---|
| | | | [dyne/cm²] | [dyne/cm²] | [η*] (P) | [dyne/cm²] | [dyne/cm²] |
| | R/G | Tg | ω = 0.01 rad/s | ω = 0.01 rad/s | ω = 0.01 rad/s | ω = 100 rad/s | ω = 100 rad/s |
| PSA | ratio | [° C.] | (T = 30° C.) | (T = 30° C.) | (T = 30° C.) | (T = 30° C.) | (T = 30° C.) |
| Example 1 | 1.65 | 61 | 5.5 × 10⁴ | 1.28 × 10⁵ | 1.39 × 10⁷ | 1.65 × 10⁷ | 1 × 10⁷ |
| Example 2 | 1.5 | 35.9 | 1.95 × 10⁴ | 1.95 × 10⁴ | 2.4 × 10⁶ | 7.88 × 10⁴ | 6.66 × 10⁴ |
| Example 3 | 1.5 | — | 3.12 × 10⁴ | 3.7 × 10⁴ | 4.39 × 10⁶ | 5.9 × 10⁶ | 6.2 × 10⁶ |
| Example 4 | 1.65 | 56.4 | 8.27 × 10⁴ | 2.23 × 10⁵ | 2.38 × 10⁷ | 2.18 × 10⁷ | 1.16 × 10⁷ |
| Example 5 | 1.65 | 63.4 | 6.57 × 10⁴ | 1.33 × 10⁵ | 1.48 × 10⁷ | 2.55 × 10⁷ | 1.3 × 10⁷ |
| Example 6 | 1.35 | 44 | 5.26 × 10⁴ | 6 × 10⁴ | 8 × 10⁶ | 7.5 × 10⁶ | 5.28 × 10⁶ |
| Example 7 | 1.35 | 64.5 | 3.78 × 10⁴ | 7.24 × 10⁴ | 8.17 × 10⁶ | 7.67 × 10⁶ | 4.3 × 10⁶ |
| Example 8 | 1.35 | 30.3 | 4.91 × 10⁴ | 9.2 × 10⁴ | 1.05 × 10⁷ | 3.33 × 10⁶ | 2 × 10⁶ |
| Example 9 | 1.65 | 64.7 | 4.59 × 10⁵ | 7.16 × 10⁵ | 8.5 × 10⁷ | 2.86 × 10⁷ | 1.16 × 10⁷ |
| Example 10 | 1.65 | 66.7 | 8.15 × 10⁴ | 1.73 × 10⁵ | 1.9 × 10⁷ | 1 × 10⁷ | 5.7 × 10⁶ |
| Example 11 | 1.65 | 18.1 | 1.28 × 10⁴ | 2.8 × 10⁴ | 2.7 × 10⁶ | 7.87 × 10⁵ | 3.47 × 10⁶ |
| Comparative | 1.35 | 82 | 3.4 × 10⁴ | 8.1 × 10⁴ | 6.27 × 10⁶ | 1.56 × 10⁷ | 1.05 × 10⁷ |
| Example 1 | | | | | | | |

Moisture Uptake from DSC

Experimental Method: PSA sample was coated on a release liner, which is a fluoro-polymer coated PET foil. The foil was dried at 100° C. for 8 hours. The PSA coated foil was then immersed in a boiling water bath for 10 minutes. The coating surface was blow dried to remove surface moisture, The PSA coated foil was then analyzed using DSC to quantitatively estimate the water uptake.

TABLE 5
Water Uptake and Water Contact Angle

| | Hydrophilic | Water | Water |
|---|---|---|---|
| | Functional Unit | Uptake | Contact Angle |
| Examples | (%) | (%) | (°) |
| Example-6 | 1.3 | 0.1872 | 93.8 |
| Example-7 | 2.61 | 2.1181 | 104.3 |
| Example-8 | 5.22 | 1.7175 | 106.6 |
| Example-9 | 0.39 | 0 | 101.1 |
| Example-10 | 1.16 | 0.134 | 95.2 |
| Example-11 | 2.33 | 3.2684 | 103.3 |
| CE-1 | 0 | 0 | 106.1 |

What has been described above includes examples of the present specification. It is, of course, not possible to describe every conceivable combination of components or methodologies for purposes of describing the present specification, but one of ordinary skill in the art may recognize that many further combinations and permutations of the present specification are possible. Accordingly, the present specification is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

The foregoing description identifies various, non-limiting embodiments of a silicone composition, adhesives formed from such compositions, and articles employing such compositions or adhesives. Modifications may occur to those skilled in the art and to those who may make and use the invention. The disclosed embodiments are merely for illustrative purposes and not intended to limit the scope of the invention or the subject matter set forth in the claims.

1. A siloxane comprising a hydrophilic group, the siloxane of the formula:
M¹ₐM²_{b}M³_{c}D¹_{d}D²ₑD³_{f}T¹_{g}T²ₕT³ₗQ¹ⱼQ²ₖQ³ₗQ⁴ₒQ⁵ₚ
where
M¹=R⁵R⁶R⁷SiO_{1/2}
M²=R⁸R⁹R¹⁰SiO_{1/2}
M³=R¹¹R¹²R¹³SiO_{1/2}
D¹=R¹⁴R¹⁵SiO_{2/2}
D²=R¹⁶R¹⁷SiO_{2/2}
D³=R¹⁸R¹⁹SiO_{2/2}
T¹=R²⁰SiO_{3/2}
T²=R²¹SiO_{3/2}
T³=R²²SiO_{3/2}
Q¹=Si(OR²³)₁O_{3/2}
Q²=Si(OR²⁴)₂O_{2/2}
Q³=Si(OR²⁵)₃O_{1/2}
Q⁴=Si(OR²⁶)₄
Q⁵=SiO_{4/2}
where:
a, b, c, d, e, f, g, h, i, j, k, l, o, and p are zero or any positive integer subject to the following limitations:
$$1 \leq [ a + b + c + d + e + f + g + h + i + j + k + l + o + p ] \leq 6000 ;$$
$$[ b + e + h + k ] \geq 1 ;$$
R⁵, R⁶, R⁷, R⁹, R¹⁰, R¹², R¹³, R¹⁴, R¹⁵, R¹⁷, R¹⁹, and R²⁰ are each independently selected from hydrogen, a C1-C60 alkyl, a C6-C60 aromatic containing group, a C1-C10 alkoxy, and a hydroxyl;
R⁸, R¹⁶, R²¹, and R²⁴ are each independently selected from a monovalent hydrophilic group of the formula, where G≥0. 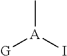
where A is a bridging unit between the siloxane moiety and the hydrophilic group I, having at least one spacing atom selected from a C1-C60 acyclic hydrocarbon radical, a C4-C60 alicylic radical, an C6-C60 aromatic hydrocarbon radical, a polyetheramine, a polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof, I is an ionic, ionizable, zwitterionic group, or a polar hydrophilic group or moieties including but not limited to a polyetheramine, a polyether, a polyamide, a polyester, a polyurethane, a polysulfone, or a polycarbonate;
G is a polymerizable group having the general Formula: 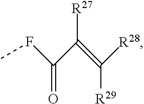
a non-polymerizable group of formula —F—K, wherein F is a linker group chosen from a C1-C60 acyclic hydrocarbon radical, a C4-C60 alicylic radical, an C6-C60 aromatic hydrocarbon radical, a polyetheramine, a polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof,
R²⁷, R²¹, and R²⁹ are independently chosen from hydrogen or monovalent hydrocarbon radical with 1-5 carbon atoms, and
K is independently chosen from hydrogen, a C1-C60 acyclic hydrocarbon radical, a C4-C60 alicylic radical, an C6-C60 aromatic hydrocarbon radical, an epoxy, or an ether group;
R¹¹, R¹⁸, R²² are each independently selected from the group -A-G where A and G are as described above; and
R²³, R²¹, and R²⁶ can be independently selected from any of R⁵ to R²² or a combination of thereof.

2. The siloxane of claim 1, wherein the hydrophilic group is a polyetheramine group selected from a compound of the formula:
R³⁶—(O—R³⁷—)_{q}—N²
where R³⁶ is selected from a C1-C60 hydrocarbon, R³⁷ is selected from a C1-C60 hydrocarbon, and q is ≥1.

3. The siloxane of claim 1, wherein the polyetheramine is selected from a compound of the formula:
R³⁶—(O—CH₂CH₂)ₓ—(O—CCH₂CH₂CH₂)_{y}—(O CH₂CH₂CH(CH₃))_{z}—NH₂
and x, y, and z are independently 0 to about 300, where x+y+z is ≥2.

4. The siloxane of claim 2, wherein the polyetheramine group is selected by the following general formula

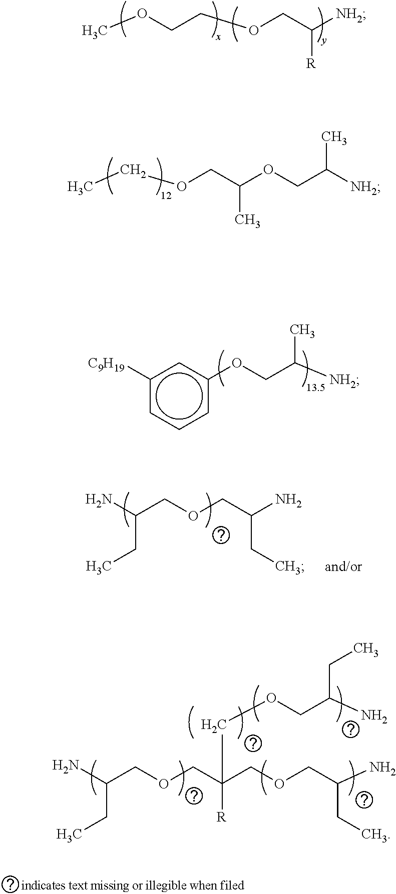

5. The siloxane of claim 1, wherein the siloxane comprising a hydrophilic group is of the formula M¹ₐM²_{b}M³_{c}Q⁵ₚ, where b is ≥1.

6. The siloxane of claim 1, wherein I is selected from a carboxylate —COO⁻, a dicarboxylate (—R(COO—)₂) a sulfone —SO₂—, a sulfonate —SO₃⁻, a sulfate —OSO₃⁻, a phosphonate —PO₃²⁻, a phosphate —OPO₃²⁻ group, —N⁺R³⁰R³¹H, —N⁺H₂R³², —N⁺H₃, or an ammonium salt, each containing a hydrogen or a cation independently selected from an alkali metal, an alkali earth metal, a transition metal, a quaternary ammonium group, and a phosphonium group, where R³⁰, R³¹, and R³² are independently selected from a C1-C30 hydrocarbon.

7. The composition of claim 1, wherein I is selected from a group having the formula —R³³—N⁺(R³⁴)₂—R³⁵—I^{z}, where R³³ is a divalent hydrocarbon group having from 1 to 20 carbon atoms, R³⁴ is a monovalent hydrocarbon group having from 1 to 20 carbon atoms, R³⁵ is a divalent hydrocarbon group having from 2 to 20 carbon atoms; and I^{z} is an ionic group selected from a carboxylate —COO⁻, a sulfone —SO₂—, a sulfonate —SO₃⁻, a sulfate —OSO₃⁻, a phosphonate —PO₃²⁻, and a phosphate —OPO₃²⁻ group.

8. A composition for forming a silicone pressure sensitive adhesive comprising (i) a MQ silicone resin, (ii) a polyorganosiloxane, and (iii) a siloxane comprising a hydrophilic group.

9. The composition of claim 8, wherein the siloxane comprises a hydrophilic functional group, wherein the hydrophilic functional group is selected from an ionic group, an ionizable group, a zwitterionic group, a polyetheramine group, or a combination of two or more thereof.

10. The composition of claim 8, wherein the siloxane comprising a hydrophilic group (iii) is selected from a compound of the formula:
M¹ₐM²_{b}M³_{c}D¹_{d}D²ₑD³_{f}T¹_{g}T²ₕT³ₗQ¹ⱼQ²ₖQ³ₗQ⁴ₒQ⁵ₚ
where
M¹=R⁵R⁶R⁷SiO_{1/2}
M²=R⁸R⁹R¹⁰SiO_{1/2}
M³=R¹¹R¹²R¹³SiO_{1/2}
D¹=R¹⁴R¹⁵SiO_{2/2}
D²=R¹⁶R¹⁷SiO_{2/2}
D³=R¹⁸R¹⁹SiO_{2/2}
T¹=R²⁰SiO_{3/2}
T²=R²¹SiO_{3/2}
T³=R²²SiO_{3/2}
Q¹=Si(OR²³)₁O_{3/2}
Q²=Si(OR²⁴)₂O_{2/2}
Q³=Si(OR²⁵)₃O_{1/2}
Q⁴=Si(OR²⁶)₄
Q⁵=SiO_{4/2}
where:
a, b, c, d, e, f, g, h, i, j, k, l, o, and p are zero or any positive integer subject to the following limitations:
$$1 \leq [ a + b + c + d + e + f + g + h + i + j + k + l + o + p ] \leq 6000 ;$$
$$[ b + e + h + k ] \geq 1 ;$$
R⁵, R⁶, R⁷, R⁹, R¹⁰, R¹², R¹³, R¹⁴, R¹⁵, R¹⁷, R¹⁹, and R²⁰ are each independently selected from hydrogen, a C1-C60 alkyl, a C6-C60 aromatic containing group, a C1-C10 alkoxy, and a hydroxyl;
R⁸, R¹⁶, R²¹, and R²⁴ are each independently selected from a monovalent hydrophilic group of the formula, where G≥0. 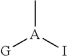
where A is a bridging unit between the siloxane moiety and the hydrophilic group I, having at least one spacing atom selected from a C1-C60 acyclic hydrocarbon radical, a C4-C60 alicylic radical, an C6-C60 aromatic hydrocarbon radical, a polyetheramine, a polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof, I is an ionic, ionizable, zwitterionic group, or a polar hydrophilic group;
G is a polymerizable group having the general Formula: 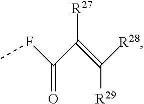
a non-polymerizable group of formula —F—K, wherein F is a linker group chosen from a C1-C60 acyclic hydrocarbon radical, a C4-C60 alicylic radical, an C6-C60 aromatic hydrocarbon radical, a a polyetheramine, polyether, a polyamide, a polyester, a polyurethane, a polysulfone, a polycarbonate, or a combination of two or more thereof, where the bridging unit may include one or more moieties selected from alkoxy, carboxy, urethane, urea, ketone, or a combination of two or more thereof,
R²⁷, R²¹, and R²⁹ are independently chosen from hydrogen or monovalent hydrocarbon radical with 1-5 carbon atoms, and
K is independently chosen from hydrogen, a C1-C60 acyclic hydrocarbon radical, a C4-C60 alicylic radical, an C6-C60 aromatic hydrocarbon radical, an epoxy, or an ether group;
R¹¹, R¹⁸, R²² are each independently selected from the group -A-G where A and G are as described above; and
R²³, R²⁵, and R²⁶ can be independently selected from R⁵ to R²² or a combination of thereof.

11. The composition of claim 10, wherein the siloxane comprising a hydrophilic group is of the formula M¹ₐM²_{b}M³_{c}Q⁵ₚ, where b is ≥1.

12. The composition of claim 10, wherein I is selected from a carboxylate —COO⁻, a dicarboxylate (—R(COO—)₂) a sulfone —SO₂—, a sulfonate —SO₃⁻, a sulfate —OSO⁻, a phosphonate —PO₃²⁻, a phosphate —OPO₃²⁻ group, —N⁺R³⁰R³¹H, —N⁺H₂R³², —N⁺H₃, or an ammonium salt, each containing a hydrogen or a cation independently selected from an alkali metal, an alkali earth metal, a transition metal, a quaternary ammonium group, and a phosphonium group, where R³⁰, R³¹, and R³² are independently selected from a C1-C30 hydrocarbon.

13. The composition of claim 10, wherein I is selected from a group having the formula —R³³—N⁺(R³⁴)₂—R³⁵—I^{z}, where R³³ is a divalent hydrocarbon group having from 1 to 20 carbon atoms, R³⁴ is a monovalent hydrocarbon group having from 1 to 20 carbon atoms, R³⁵ is a divalent hydrocarbon group having from 2 to 20 carbon atoms; and I^{z} is an ionic group selected from a carboxylate —COO⁻, a sulfone —SO₂—, a sulfonate —SO₃⁻, a sulfate —OSO₃⁻, a phosphonate —PO₃²⁻, and a phosphate —OPO₃²⁻ group.

14. The composition of claim 10, wherein I is a polar group selected from a polyetheramine, a polyether, a polyamide, a polyester, a polyurethane, a polysulfone, and/or a polycarbonate

15. The composition of claim 10, wherein the hydrophilic group is a polyetheramine group selected from a compound of the formula
R³⁶—(O—R³⁷—)_{q}—NH₂
where R³⁶ is selected from a C1-C60 hydrocarbon, R³⁷ is selected from a C1-C60 hydrocarbon, and q is ≥1.

16. The composition of claim 15, wherein the polyetheramine is selected from a compound of the formula:
R³⁶—(O—CH₂CH₂)ₓ—(O—CCH₂CH₂CH₂)_{y}—(O CH₂CH₂CH(CH₃))_{z}—NH₂
and x, y, and z are independently 0 to about 300, where x+y+z is ≥2.

17. The composition of claim 9, wherein the polyetheramine group is selected by the following general formula

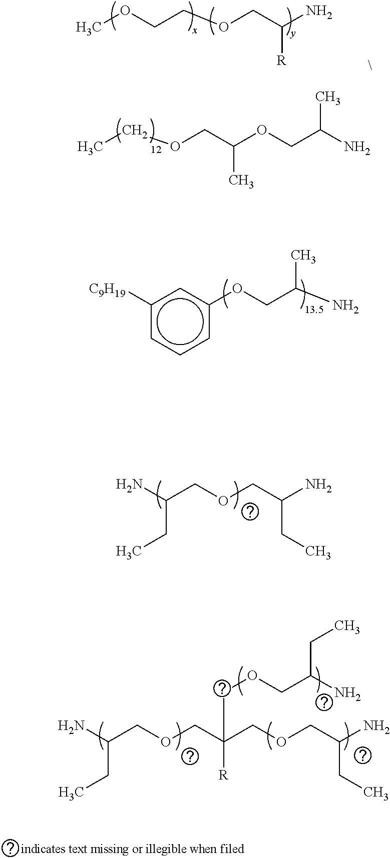

18. The composition of claim 8, wherein the siloxane comprising a hydrophilic group (iii) is present in an amount of from about 0.1 wt. % to about 20 wt. % based on the total weight of the composition.

19. The composition of claim 8, wherein the siloxane comprising a hydrophilic group (iii) is present in an amount of from about 1.5 wt. % to about 8 wt. % based on the total weight of the composition.

20. A pressure sensitive adhesive formed from the composition of claim 8.

21. The pressure sensitive adhesive of claim 20, wherein the adhesive has a less than 2500 ppm of one or more of octamethylcyclotetrasiloxane, decamethylcyclopentasiloxane, and/or dodecamethylcyclohexasiloxane.

22. The pressure sensitive adhesive of claim 20, wherein the adhesive has a less than 1000 ppm of one or more of octamethylcyclotetrasiloxane, decamethylcyclopentasiloxane, and/or dodecamethylcyclohexasiloxane.

23. The pressure sensitive adhesive of claim 20, wherein the adhesive has a less than 500 ppm of one or more of octamethylcyclotetrasiloxane, decamethylcyclopentasiloxane, and/or dodecamethylcyclohexasiloxane.

24. The pressure sensitive adhesive of claim 20, wherein the adhesive has a less than 100 ppm of one or more of octamethylcyclotetrasiloxane, decamethylcyclopentasiloxane, and/or dodecamethylcyclohexasiloxane.

25. An article comprising the pressure sensitive adhesive of claim 20.

26. The article of claim 25, wherein the article is selected from a wound dressing, a dressing for scar reduction, a drug delivery device, a medical tubing, a clinical surface, a pacemaker lead, a wound healing patch, a wound management device, a catheter, a shunt, a valve, a stent, a transdermal patch, a scaffold for tissue engineering, an anti-microbial device, a ophthalmic device, a bioinsert, a plug, a surgical device, an assisted breathing apparatus, a prosthetic device, a reconstructive device, or a body implant.

27. The article of claim 25, wherein the article is selected from a cream, a lotions, a bioactive compound, deodorant, an insecticide, a decorative cosmetic, lipstick, eye shadow, stage make-up, a hand cleaner, a face mask, a pore cleanser, a knee- or elbow-protector, a bra, a surgical gown, a nasal plasters; a prosthesis, a cold wrap, a thermal wrap to a hearing aid; a protective face masks, an anti-snoring patch, jewelry, earrings, guises, tattoos, eye wear, a tape, or a bandage.
